(19) United States
(12) Patent Application Publication
(10) Pub. No.: US 2025/0243542 A1
(43) Pub. Date: Jul. 31, 2025

(54) METHOD OF COMPARING GENETIC IDENTITY AND METHOD OF DETERMINING IDENTITY OF SOURCE SUBJECT OF SPECIMENS

(71) Applicant: Chun-Hsuan HO, Taipei City (TW)

(72) Inventor: Chun-Hsuan HO, Taipei City (TW)

(21) Appl. No.: 18/803,848

(22) Filed: Aug. 14, 2024

(30) Foreign Application Priority Data
Jan. 31, 2024 (TW) 113103853

(51) Int. Cl. C12Q 1/6881; C12Q 1/6806; C12Q 1/6844; C12Q 1/6869

(52) U.S. Cl. CPC C12Q 1/6881; C12Q 1/6806; C12Q 1/6844; C12Q 1/6869

(57) ABSTRACT

A method of comparing genetic identity is provided in some embodiments of the present disclosure, including comparing genetic identity of human leukocyte antigen (HLA) genes, short tandem repeats (STR), killer-cell immunoglobulin-like receptor (KIR) genes of a test specimen with a target gene group, and wherein when a comparison result of HLA genes, a comparison result of STR and a comparison result of KIR genes are all identical, the test gene group of the test specimen is determined to be identical to the target gene group. A method of determining identity of source subjects of specimens is provided in some embodiments of the present disclosure.

### CROSS-REFERENCE TO RELATED APPLICATION

This application claims priority to Taiwan Application Serial Number 113103853, filed Jan. 31, 2024, which is herein incorporated by reference.

### BACKGROUND

### Field of Invention

The present disclosure is related to a method of comparing genetic identity and a method of determining identity of source subjects of multiple specimens.

### Description of Related Art

During cell therapy or organ transplantation, the higher the genetic identity between the effector cells and the original subject cells, the lower the risk of immune rejection. When the effector cells and the original subject cells originate from the same subject, the risk of immune rejection can be minimized. Therefore, when a subject is in a healthy state, subject cells can be cryopreserved in advance for future use when cell therapy or organ transplantation is needed. However, since cell cryopreservation banks usually store a large number of cells originating from different subjects at the same time, once the wrong effector cells are used for treatment, it can easily lead to severe immune rejection in the subject.

Therefore, it is necessary to confirm whether the effector cell (hereinafter referred to as test specimen) and the original subject cell (hereinafter referred to as target specimen) originates from the same source subject before treatment, so that subsequent treatment can be continued so as to reduce immune rejection.

However, the current gene comparison method has limited accuracy (for example, by using short tandem repeats (STR), if the genes of different specimens match more than 80%, the specimens are determined to be originated from the same subject), and there is still considerable risk of misjudgments.

Therefore, how to provide a method for increasing accuracy of comparing genetic identity and determining source subjects of specimens is a problem to be solved.

### SUMMARY

Some embodiments of the present disclosure provides a method of comparing genetic identity, including: providing a target gene group; providing a test specimen including a first test specimen, a second test specimen and a third test specimen; comparing an identity between human leukocyte antigen (HLA) genes of the first test specimen and human leukocyte antigen genes of the target gene group to obtain a comparison result of human leukocyte antigen genes; comparing an identity between short tandem repeats (STR) of the second test specimen and short tandem repeats of the target gene group to obtain a comparison result of short tandem repeats; comparing an identity between killer-cell immunoglobulin-like receptor (KIR) genes of the third test specimen and killer-cell immunoglobulin-like receptor genes of the target gene group to obtain a comparison result of killer-cell immunoglobulin-like receptor genes; and wherein when the comparison result of human leukocyte antigen genes, the comparison result of short tandem repeats and the comparison result of killer-cell immunoglobulin-like receptor genes are all identical, the target gene group is determined to be identical to a test gene group of the test specimen.

In some embodiments, the first test specimen includes blood.

In some embodiments, the step of comparing the identity between the human leukocyte antigen genes of the first test specimen and the human leukocyte antigen genes of the target gene group includes: extracting and amplifying the human leukocyte antigen genes of the first test specimen; and analyzing and comparing the identity between the human leukocyte antigen genes of the first test specimen and the human leukocyte antigen genes of the target gene group by using next generation sequencing.

In some embodiments, the step of comparing the identity between the human leukocyte antigen genes of the first test specimen and the human leukocyte antigen genes of the target gene group includes: comparing genetic the identity of HLA-A, HLA-B, HLA-C, HLA-DRB1, HLA-DQB1, or a combination thereof.

In some embodiments, the step of comparing the identity between the human leukocyte antigen genes of the first test specimen and the human leukocyte antigen genes of the target gene group includes: comparing an identity between a coding region of the human leukocyte antigen genes of the first test specimen and a coding region of the human leukocyte antigen genes of the target gene group; and comparing an identity between a non-coding region of the human leukocyte antigen genes of the first test specimen and a non-coding region of the human leukocyte antigen genes of the target gene group.

In some embodiments, the second test specimen includes human tissue cells.

In some embodiments, the step of comparing the identity between the short tandem repeats of the second test specimen and the short tandem repeats of the target gene group includes: extracting the short tandem repeats of the second test specimen; and analyzing and comparing the identity between the short tandem repeats of the second test specimen and the short tandem repeats of the target gene group by using sequencing-based typing.

In some embodiments, the step of comparing the identity between the short tandem repeats of the second test specimen and the short tandem repeats of the target gene group includes: comparing genetic identity of gene locus D8S1179, D21S11, D7S820, CSF1PO, D3S1358, TH01, D13S317, D16S539, D2S1338, D19S433, vWA, TPOX, D18S51, Amelogenin, D5S818, FGA, or a combination thereof.

In some embodiments, the third test specimen includes blood.

In some embodiments, the step of comparing the identity between the killer-cell immunoglobulin-like receptor genes of the third test specimen and the killer-cell immunoglobulin-like receptor genes of the target gene group includes: extracting the killer-cell immunoglobulin-like receptor genes of the third test specimen; and analyzing and comparing the identity between the killer-cell immunoglobulin-like receptor genes of the third test specimen and the killer-cell immunoglobulin-like receptor genes of the target gene group by using polymerase chain reaction-sequence-specific oligonucleotides probes (PCR-SSOP), polymerase chain reaction-sequence-specific primers (PCR-SSP), sequencing-based typing (SBT), or a combination thereof.

In some embodiments, the step of comparing the identity between the killer-cell immunoglobulin-like receptor genes of the third test specimen and the killer-cell immunoglobulin-like receptor genes of the target gene group includes: comparing genetic identity of 2DL1, 2DL2, 2DL3, 2DL4, 2DL5, 2DS1, 2DS2, 2DS3, 2DS4, 2DS5, 3DL1, 3DL2, 3DL3, 3DS1, 2DP1, 3DP1, or a combination thereof.

Some embodiments of the present disclosure provides a method of determining identity of a plurility of source subjects of a plurility of specimens, including: providing a test specimen and a target specimen; comparing an identity between human leukocyte antigen genes of the test specimen and human leukocyte antigen genes of the target specimen to obtain a comparison result of human leukocyte antigen genes; comparing an identity between short tandem repeats of the test specimen and short tandem repeats of the target specimen to obtain a comparison result of short tandem repeats; comparing an identity between killer-cell immunoglobulin-like receptor genes of the test specimen and killer-cell immunoglobulin-like receptor genes of the target specimen to obtain a comparison result of killer-cell immunoglobulin-like receptor genes; and wherein when the comparison result of human leukocyte antigen genes, the comparison result of short tandem repeats and the comparison result of killer-cell immunoglobulin-like receptor genes are all identical, a source subject of the test specimen is determined to be identical to a source subject of the target specimen.

In some embodiments, the test specimen and the target specimen include blood.

In some embodiments, the step of comparing the identity between the human leukocyte antigen genes of the test specimen and the human leukocyte antigen genes of the target specimen includes: extracting and amplifying the human leukocyte antigen genes of the test specimen and the human leukocyte antigen genes of the target specimen; and analyzing and comparing the identity between the human leukocyte antigen genes of the test specimen and the human leukocyte antigen genes of the target specimen by using next generation sequencing.

In some embodiments, the step of comparing the identity between the human leukocyte antigen genes of the test specimen and the human leukocyte antigen genes of the target specimen includes: comparing genetic identity of HLA-A, HLA-B, HLA-C, HLA-DRB1, HLA-DQB1, or a combination thereof.

In some embodiments, the step of comparing the identity between the human leukocyte antigen genes of the test specimen and the human leukocyte antigen genes of the target specimen includes: comparing the identity between a coding region of the human leukocyte antigen genes of the test specimen and a coding region of the human leukocyte antigen genes of the target specimen; and comparing the identity between a non-coding region of the human leukocyte antigen genes of the test specimen and a non-coding region of the human leukocyte antigen genes of the target specimen.

In some embodiments, the step of comparing the identity between the short tandem repeats of the test specimen and the short tandem repeats of the target specimen includes: extracting the short tandem repeats of the test specimen and the short tandem repeats of the target specimen; and analyzing and comparing the identity between the short tandem repeats of the test specimen and the short tandem repeats of the target specimen by using sequencing-based typing.

In some embodiments, the step of comparing the identity between the short tandem repeats of the test specimen and the short tandem repeats of the target specimen includes: comparing genetic identity of gene locus D8S1179, D21S11, D7S820, CSF1PO, D3S1358, TH01, D13S317, D16S539, D2S1338, D19S433, vWA, TPOX, D18S51, Amelogenin, D5S818, FGA, or a combination thereof.

In some embodiments, the step of comparing the identity between the killer-cell immunoglobulin-like receptor genes of the test specimen and the killer-cell immunoglobulin-like receptor genes of the target specimen, including: extracting the killer-cell immunoglobulin-like receptor genes of the test specimen and the killer-cell immunoglobulin-like receptor genes of the target specimen; and analyzing and comparing the identity between the killer-cell immunoglobulin-like receptor genes of the test specimen and the killer-cell immunoglobulin-like receptor genes of the target specimen by using polymerase chain reaction-sequence-specific oligonucleotides probes, polymerase chain reaction-sequence-specific primers, sequencing-based typing, or a combination thereof.

In some embodiments, the step of comparing the identity between the killer-cell immunoglobulin-like receptor genes of the test specimen and the killer-cell immunoglobulin-like receptor genes of the target specimen includes comparing genetic identity of 2DL1, 2DL2, 2DL3, 2DL4, 2DL5, 2DS1, 2DS2, 2DS3, 2DS4, 2DS5, 3DL1, 3DL2, 3DL3, 3DS1, 2DP1, 3DP1, or a combination thereof.

### BRIEF DESCRIPTION OF THE DRAWINGS

In order to allow the above-mentioned and other purposes, features, advantages and embodiments of the present disclosure to be more clearly understood, accompanying drawing is described as follows:

FIG. 1 illustrates a flow chart of a method of comparing genetic identity in some embodiments of the present disclosure.

FIG. 2 illustrates a flow chart of a method of determining identity of source subjects of specimens in some embodiments of the present disclosure.

### DETAILED DESCRIPTION

It is to be understood that different implementations or embodiments provided in the following may implement different features of the subject matter of the present disclosure. The embodiments of specific components and arrangements are used to simplify the disclosure and not to limit the disclosure. Of course, these are only examples and are not intended to be limiting. For example, the description below that the first feature is formed on the second feature includes the two being in direct contact, or there are other additional features between the two that are not in direct contact. Furthermore, the present disclosure may repeat reference numerals and/or symbols in the various embodiments. Such repetition is for simplicity and clarity and does not represent a relationship between the various embodiments and/or configurations discussed.

As used herein, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

Although a series of operations or steps are described below to illustrate the method disclosed herein, the order of the operations or steps is not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein can include a plurality of sub-steps or actions.

As used herein, polymerase chain reaction-sequence-specific oligonucleotide method (PCR-SSOP) indicates hybridizing probes labeled with an isotope or non-radioactive label with fragments of the test gene group amplified by polymerase chain reaction (PCR). The match signals of hybridization are used to determine the genotype of the test gene group.

As used herein, polymerase chain reaction-sequence-specific primers (PCR-SSP) indicates that the gene is amplified with specific primers by using PCR, and then the genotype of the test gene group is determined based on whether the gene is amplified or not.

As used herein, sequencing-based typing (SBT) indicates a method to directly sequence genes, such as Sanger SBT (SSBT) or next generation sequencing (Next Generation Sequencing, NGS).

Some embodiments of the present disclosure compare genetic identity of human leukocyte antigen (HLA) genes, short tandem repeats (STR) and killer-cell immunoglobulin-like receptor (KIR) between a test specimen and a target specimen, determine if the target gene group and the test gene group are identical, and determine if the test specimen and the target specimen originate from the same source subject. Through the comparisons of triple highly polymorphic genes, the test specimen and the target specimen are likely locked by genetic code lock, which minimizes the probability of getting the wrong test specimen (the probability that the three results are all identical is 1/10 billion).

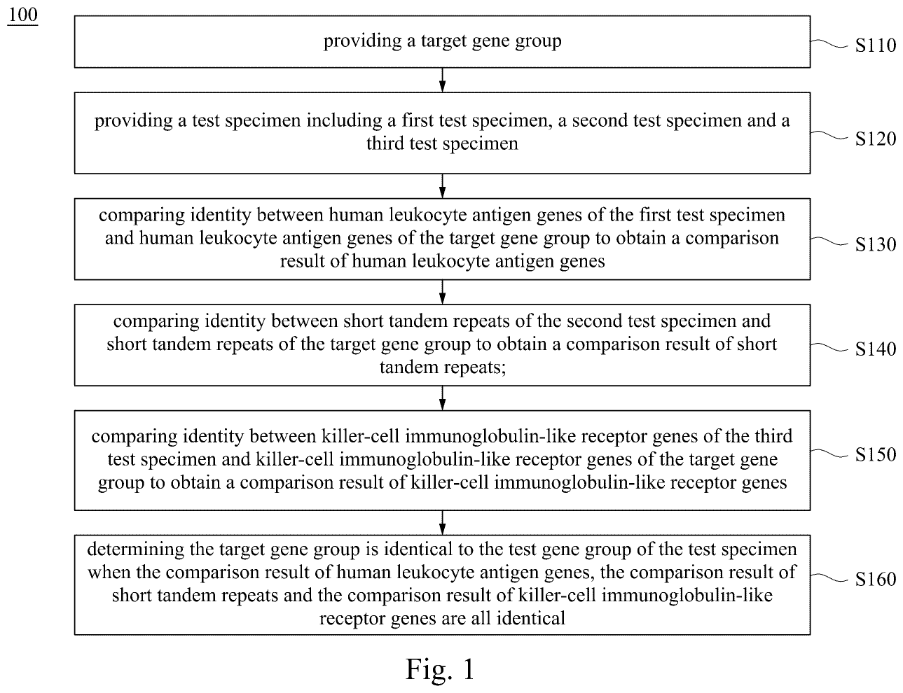

First of all, please refer to FIG. 1, a method 100 of comparing genetic identity is provided, including step S110, step S120, step S130, step S140, step S150 and step S160.

Step S110, a target gene group is provided.

In some embodiments, the target gene group is gene sequence of the target specimen, including human HLA genes, STR and KIR genes. In some embodiments, HLA genes of the target gene group include genes of HLA-A, HLA-B, HLA-C, HLA-DRB1 and HLA-DQB1. In some embodiments, STR of the target gene group include sequence of gene locus D8S1179, D21S11, D7S820, CSF1PO, D3S1358, TH01, D13S317, D16S539, D2S1338, D19S433, vWA, TPOX, D18S51, Amelogenin, D5S818 and FGA. In some embodiments, KIR genes of the target gene group include genes of 2DL1, 2DL2, 2DL3, 2DL4, 2DL5, 2DS1, 2DS2, 2DS3, 2DS4, 2DS5, 3DL1, 3DL2, 3DL3, 3DS1, 2DP1 and 3DP1. It should be emphasized that the gene sequences of the abovementioned HLA, STR and KIR are fragments with higher polymorphism in HLA, STR and KIR, respectively, which can decrease the probability that the genes of other specimens are all identical to the target gene group during the further comparison (about 1/10 billion) and improve accuracy of comparison. In some embodiments, the target specimen includes blood, human tissue cells, or a combination thereof.

Step S120, a test specimen is provided, including a first test specimen, a second test specimen and a third test specimen.

In some embodiments, the first test specimen includes blood (mainly used for providing lymphocytes) for analyzing HLA genes. In some embodiments, the second test specimen includes human tissue cells for analyzing STR. For example, the human tissue cells include oral cavity cells, blood, hair, placenta, umbilical cord, amniotic fluid, saliva, or a combination thereof. In some embodiments, the third test specimen includes blood (mainly used for providing natural killer cells) for analyzing KIR genes. The type of the test specimen basically corresponds to the type of the target specimen.

Step S130, identity between HLA genes of the first test specimen and HLA genes of the target gene group are compared to obtain a comparison result of HLA genes.

In some embodiments, step S130 includes extracting and amplifying HLA genes of the first test specimen; and analyzing and comparing identity between the HLA genes of the first test specimen and the HLA genes of the target gene group by using NGS.

In some embodiments, by using NGS, HLA resolution can reach 8-digit high resolution. Specifically, the 1st digit and the 2nd digit (zone 1) represent HLA serology typing or gene groups of allele genes, and the 3rd digit and the 4th digit (zone 2) represent allele genes with amino acid variations in the coding region, and the 5th and the 6th digits (zone 3) represent allele genes with no amino acid variations in the coding region (synonymous DNA substitution), the 7th digit and the 8th digit (zone 4) represent the allele genes with base substitution in the non-coding region.

That is, step S130 includes comparing identity between coding regions in HLA genes of the first test specimen and coding regions in HLA genes of the target gene group; and comparing identity between non-coding regions in HLA genes of the first test specimen and non-coding regions in HLA genes of the target gene group. It should be emphasized that, compared with resolution only to the coding region, further analyzing and comparing gene sequence of the non-coding region can not only increase identification accuracy of genetic identity, but also take the comparison result of the non-coding region into reference, which improves the success rate of treatment when performing cell therapy or organ transplantation.

In some embodiments, step S130 includes comparing genetic identity of HLA-A, HLA-B, HLA-C, HLA-DRB1, HLA-DQB1, or a combination thereof, which are subtypes with higher polymorphism. It can be understood that, HLA genes are the gene types with highest polymorphism in human genes. The accuracy of gene comparison can be increased by selecting abovementioned subtypes with higher polymorphism in HLA genes.

In some embodiments, the comparison result of HLA genes represents whether each subtype of HLA genes of the first test specimen is identical to each subtype of HLA genes of the target gene group. When the tested results of HLA subtypes are all identical, the comparison result of HLA genes is determined to be identical.

Step S140, identity between the short tandem repeats (STR) of the second test specimen and the short tandem repeats (STR) of the target gene group is compared to obtain the comparison result of STR.

In some embodiments, step S140 includes extracting STR of the second test specimen; and analyzing and comparing identity between the STR of the second test specimen and the STR of the target gene group by using SBT. Compared with PCR-SSP or PCR-SSOP, SBT is directly sequencing genes; therefore, the higher comparison accuracy can be achieved by SBT.

In some embodiments, step S140 includes comparing genetic identity of gene locus D8S1179, D21S11, D7S820, CSF1PO, D3S1358, TH01, D13S317, D16S539, D2S1338, D19S433, vWA, TPOX, D18S51, Amelogenin, D5S818, FGA, or a combination thereof. These gene locuses are the gene locuses with higher polymorphism. It can be understood that STR are gene types with higher polymorphism in human genes. The accuracy of gene comparison can be increased by selecting abovementioned gene locuses with higher polymorphism in STR genes

Step S150, identity between KIR genes of the third test specimen and KIR genes of the target gene group is compared to obtain a comparison result of KIR genes.

In some embodiments, step S150 includes extracting KIR genes of the third test specimen; and analyzing and comparing identity between the KIR genes of the third test specimen and the KIR genes of the target gene group by using PCR-SSOP, PCR-SSP, SBT or a combination thereof.

In some embodiments, for increasing accuracy of testing, analyzing and comparing can be performed by combining multiple analyzing methods. For example, PCR-SSOP and PCR-SSP, which consume less time, can be performed first, and the bias of the respective method can be reduced by comparing whether the results of the two test methods are all identical. In another embodiment, when it is difficult to design primers for gene fragments, PCR-SSOP can be selected to be pair with SBT with higher sequence resolution to compare whether the results of the two testing methods are identical, so as to save the primer design time when using PCR-SSP and avoid the limitation of gene detection section due to the difficulty of primer design.

In some embodiments, step S150 includes comparing genetic identity of 2DL1, 2DL2, 2DL3, 2DL4, 2DL5, 2DS1, 2DS2, 2DS3, 2DS4, 2DS5, 3DL1, 3DL2, 3DL3, 3DS1, 2DP1, 3DP1, or a combination thereof. These gene fragments are the genes with higher polymorphism. It can be understood that KIR genes are gene types with high polymorphism in human genes and are highly associated with immune response. The accuracy of gene comparison can be increased and the risk of immune rejection during treatment can be decreased by selecting gene sections with higher polymorphism in KIR genes.

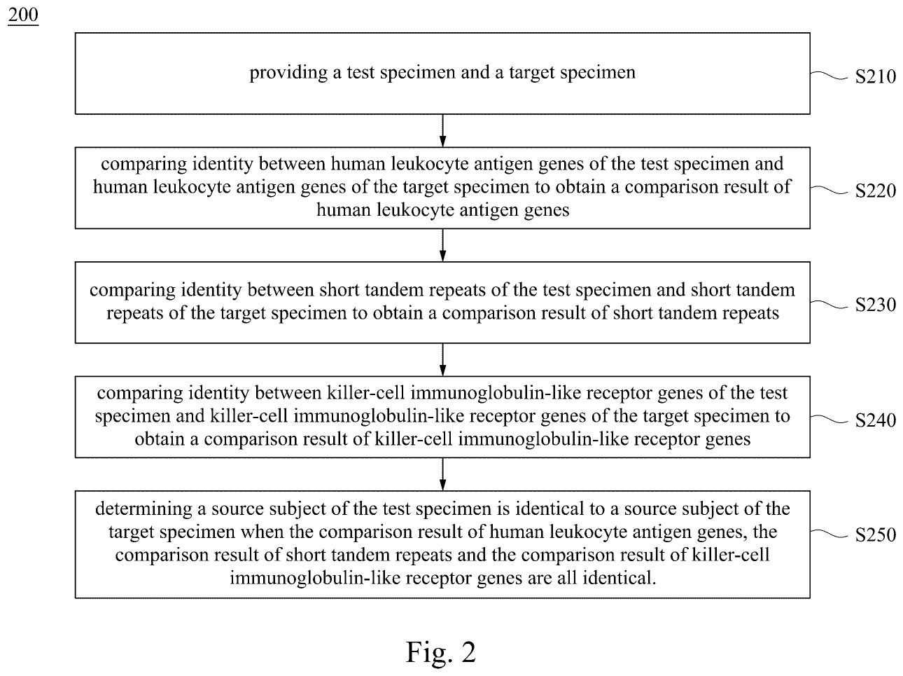

Please refer to FIG. 2, a method 200 of determining identity of a plurility of source subjects of a plurility of specimens in the present disclosure, including step S210 to step S250. Step S210, a test specimen and a target specimen are provided. In some embodiments, when the test specimen and the target specimen are blood, the analysis of HLA genes, STR and KIR genes can all be performed.

Step S220, identity between HLA genes of the test specimen and HLA genes of the target specimen is compared to obtain a comparison result of HLA genes. Step S230, identity between STR of the test specimen and STR of the target specimen is compared to obtain a comparison result of STR. Step S240, identity between KIR genes of the test specimen and KIR genes of the target specimen is compared to obtain a comparison result of KIR genes. Step S250, it is determined that a source subject of the test specimen is identical to a source subject of the target specimen when the comparison result of HLA genes, the comparison result of STR and the comparison result of KIR genes are all identical. Step S220 basically corresponds to step S130, step S230 basically corresponds to step S140, step S240 basically corresponds to step S150, step S250 basically corresponds to step S160. The method of analysis and comparison can be referred to the above and will not be described again.

According to the foregoing description, the method 200 decreases the probability that the three results are all identical to 1/10 billion by analyzing and comparing triple highly polymorphic gene types (HLA genes, STR, KIR genes) so as to increase identification accuracy. Therefore, when the comparison results of the genes are all identical, it can be determined that the source subject of the test specimen is identical to the source subject of the target specimen, which can be used for subsequent treatment.

In order to further illustrate the method of comparing genetic identity and the method of determining identity of source subjects of specimens in the various embodiments of the present disclosure, the following implementations are carried out. It should be noted that the following examples are only provided for demonstration only and not to limit the present disclosure.

The three operation procedures for determining genetic identity are listed below. Since the genes of the target specimen and the test specimen can be analyzed by the same methods, the procedure of analyzing genes of the test specimen is represented in the foregoing for simplicity. It can be understood that the gene analysis of HLA genes, STR and KIR of the target specimen can be analyzed in advance by the method the same as which of the test specimen before cell cryopreservation.

### 1. Identity of HLA Genes

HLA genes are the most polymorphic system in humans. The probability of a complete match of HLA genes is extremely low (depending on different races, the probability of a complete match of HLA genes between two subjects ranges from 1 in 2 billion to 1 in 200000). Therefore, the identification accuracy between the target specimen and the test specimen can be increased by selecting HLA genes to perform comparison of genetic identity, and the analysis procedure is listed below.

First, blood was provided for serving as the test specimen.

Furthermore, HLAssure SE HLA SBT gene typing reagent of TBG Diagnostics (HLAssure SE A Locus SBT Kit, catalog number 50110; HLAssure SE B Locus SBT Kit, catalog number 50210; HLAssure SE C Locus SBT Kit, catalog number 50410; HLAssure SE DRB1-EX Locus SBT Kit, catalog number 50350; HLAssure SE DQB1 Locus SBT Kit, catalog number 50510) was used to extract and amplify HLA genes of the test specimen. After banking HLA genes, HLA high resolution typing sequencer (brand: Applied Biosystems™; name: 3730xl DNA Analyzer; product number: A41046) was used to analyze sequences of HLA-A, HLA-B, HLA-C, HLA-DRB1 and HLA-DQB1, in which sequence resolution reached 8 digits, including gene types of coding region and non-coding region genes.

Furthermore, the genes of HLA-A, HLA-B, HLA-C, HLA-DRB1 and HLA-DQB1 between the target specimen and the test specimen were compared to determine if they were all identical to obtain a comparison result of HLA genes.

It should be noted that the resolution of NGS reflected on whether the gene sequences could be completely read. It HLA-A was taken as an example, A01 was subdivided into A01:01 (GGATCATC), A01:02 (GGATGAAG), A01:03 (GGATCTAG) . . . etc, according to sequence difference. When using low-resolution analysis (for example, 2 digits), only the first four gene sequences such as A01 (GGATXXXX), A02 (CTGGXXXX), A03 (ATCGXXXX) were compared, and the last four gene sequences were ignored for rapid detection of whether the comparison result was positive. However, using low-resolution analysis, even if HLA-A, HLA-B, HLA-C, HLA-DR, and HLA-DQ were all identical, there was still high probability of causing rejection or death in cell therapy or organ transplantation.

Therefore, compared with the sequencing resolution to only 2 or 4 digits (it was commonly known that when 4 digits were detected, similarity of more than 80% was determined to be the same source subject), the digits of NGS sequence analysis in the present disclosure were increased to 8 digits (for example, HLA-A A*01:01:01:01 was read), and the genes must be completely identical, so as to improve the accuracy of HLA sequence identification and reduce rejection in subsequent treatments.

### 2. Identity of STR

The gene position point of STR is composed of 3 base pairs to 7 base pairs in length. STR is widely present in the human genetic group and has high diversity. Therefore, the identification accuracy between the target specimen and the test specimen can be increased by selecting STR to perform comparison of identity, and the analysis procedure is listed below.

First, human tissue cells (such as oral cavity cells or blood) were provided for serving as the test specimen.

Furthermore, DNA extraction kit (brand: Invitrogen; name: PureLink™ Genomic DNA Mini Kit; product number: K182001) was used to extract STR of the test specimen.

The extracted STR was amplified by the amplification kit (brand: Applied Biosystems; name: AmpFLSTR® Identifiler® PCR Amplification Kit; product number: 4322288).

The sequences of gene locus D8S1179, D21S11, D7S820, CSF1PO, D3S1358, TH01, D13S317, D16S539, D2S1338, D19S433, vWA, TPOX, D18S51, Amelogenin, D5S818 and FGA in STR were sequenced by Sequencing-Based Typing (SBT) with sequencing analyzer machine (brand: Applied Biosystems; name: SeqStudio Genetic Analyzer; product number: A35644).

Furthermore, the genes of the abovementioned gene locus of STR in the target specimen and the test specimen were compared to determine if they were all identical to obtain the comparison result of STR.

It should be noted that, compared with other gene locus of STR, the combination of STR gene locus of the present disclosure increased identification accuracy to 99% by selecting positions with highly gene polymorphism.

### 3. Identity of KIR

KIR genes are a general term for a group of genes located on human chromosome 19. The existence of KIR genes expresses KIR proteins in natural killer cells, in which KIR protein plays important roles in human immune response. KIR genes are 17 kinds of genes in total, but not everyone express all KIR genes, and different combinations of KIR genes form different KIR gene haplotypes, so KIR genes have a high degree of gene polymorphism. In addition, in organ transplantation treatment, when HLA genes are all identical, and the recipient and donor have the same KIR gene haplotypes, the success rate of transplantation is increased and the risk of acute myeloid leukemia (AML) is reduced. Therefore, it helped improve the success rate of subsequent cell therapy or organ transplantation if all KIR genes are identical (the same source subject).

The analysis procedure is listed below.

First, blood was provided for serving as the test specimen.

Furthermore, 2DL1, 2DL2, 2DL3, 2DL4, 2DL5, 2DS1, 2DS2, 2DS3, 2DS4, 2DS5, 3DL1, 3DL2, 3DL3, 3DS1, 2DP1, 3DP1 of KIR were determined if the gene fragments intened to be detected existed in the test specimen by PCR-SSP method, in which the KIR typing kit (name: Exprobe™ KIR Typing Kit; product number: 69010) provided by TBG Biotechnology Corp. was used, and PCR was performed by sequence-specific primers with PCR machine (brand: Applied Biosystems; name: 7500 Real-Time PCR system).

It was understood that any method available to analyze the aforementioned KIR gene types could be used in the examples, including but not limited to PCR-SSP, PCR-SSOP, and SBT. In addition, multiple sequence analyzing methods could be also used at the same time in demand to improve detection accuracy.

Furthermore, the distributions of the abovementioned KIR gene blocks between the target specimen and the test specimen were compared (the sequencing results were compared if SBT method was used) to determine if they were all identical to obtain the comparison result of KIR genes.

### 4. Determination of Genetic Identity of Specimens

The probability that the target gene group matched the test gene group was as low as about 1/10 billion by triple genetic sequence comparisons of the abovementioned highly polymorphic Example 1 (HLA), Example 2 (STR) and Example 3 (KIR). Therefore, the situation that the three comparison results (HLA, STR and KIR) were all identical between the target specimen and test specimen was only one person in the population of the whole world, which was likely to lock the specimen with triple gene locks. Through extremely low probability of genetic identity, the source subjects were ensured to be the same when the genes matched between the specimens.

Therefore, according to the abovementioned comparisons of the examples, when the results between the test specimen and the target specimen matched, the test gene group of the test specimen and the target gene group of the target specimen were determined to be identical, thereby determining the source subject of the test specimen and the source subject of the target specimen were identical, and the test specimen was available to be used in the subsequent treatment.

Although the present invention has been disclosed in the above embodiments, it is not intended to limit the present invention. Anyone familiar with this technique can make various changes and modifications without departing from the spirit and scope of the present invention. The scope of protection of the invention shall be subjected to the scope of appended claims.

What is claimed is:

1. A method of comparing genetic identity, comprising:
providing a target gene group;
providing a test specimen comprising a first test specimen, a second test specimen and a third test specimen;
comparing an identity between human leukocyte antigen genes of the first test specimen and human leukocyte antigen genes of the target gene group to obtain a comparison result of human leukocyte antigen genes;
comparing an identity between short tandem repeats of the second test specimen and short tandem repeats of the target gene group to obtain a comparison result of short tandem repeats;
comparing an identity between killer-cell immunoglobulin-like receptor genes of the third test specimen and killer-cell immunoglobulin-like receptor genes of the target gene group to obtain a comparison result of killer-cell immunoglobulin-like receptor genes;
wherein when the comparison result of human leukocyte antigen genes, the comparison result of short tandem repeats and the comparison result of killer-cell immunoglobulin-like receptor genes are all identical, the target gene group is determined to be identical to a test gene group of the test specimen.

2. The method of claim 1, wherein the first test specimen comprises blood.

3. The method of claim 1, wherein the step of comparing the identity between the human leukocyte antigen genes of the first test specimen and the human leukocyte antigen genes of the target gene group comprises:
extracting and amplifying the human leukocyte antigen genes of the first test specimen; and
analyzing and comparing the identity between the human leukocyte antigen genes of the first test specimen and the human leukocyte antigen genes of the target gene group by using next generation sequencing.

4. The method of claim 1, wherein the step of comparing the identity between the human leukocyte antigen genes of the first test specimen and the human leukocyte antigen genes of the target gene group comprises: comparing genetic identity of HLA-A, HLA-B, HLA-C, HLA-DRB1, HLA-DQB1, or a combination thereof.

5. The method of claim 1, wherein the step of comparing the identity between the human leukocyte antigen genes of the first test specimen and the human leukocyte antigen genes of the target gene group comprises:
comparing an identity between a coding region of the human leukocyte antigen genes of the first test specimen and a coding region of the human leukocyte antigen genes of the target gene group; and
comparing an identity between a non-coding region of the human leukocyte antigen genes of the first test specimen and a non-coding region of the human leukocyte antigen genes of the target gene group.

6. The method of claim 1, wherein the second test specimen comprises human tissue cells.

7. The method of claim 1, wherein the step of comparing the identity between the short tandem repeats of the second test specimen and the short tandem repeats of the target gene group comprises:
extracting the short tandem repeats of the second test specimen; and
analyzing and comparing the identity between the short tandem repeats of the second test specimen and the short tandem repeats of the target gene group by using sequencing-based typing.

8. The method of claim 1, wherein the step of comparing the identity between the short tandem repeats of the second test specimen and the short tandem repeats of the target gene group comprises: comparing genetic identity of gene locus D8S1179, D21S11, D7S820, CSF1PO, D3S1358, TH01, D13S317, D16S539, D2S1338, D19S433, vWA, TPOX, D18S51, Amelogenin, D5S818, FGA, or a combination thereof.

9. The method of claim 1, wherein the third test specimen comprises blood.

10. The method of claim 1, wherein the step of comparing the identity between the killer-cell immunoglobulin-like receptor genes of the third test specimen and the killer-cell immunoglobulin-like receptor genes of the target gene group comprises:
extracting the killer-cell immunoglobulin-like receptor genes of the third test specimen; and
analyzing and comparing the identity between the killer-cell immunoglobulin-like receptor genes of the third test specimen and the killer-cell immunoglobulin-like receptor genes of the target gene group by using polymerase chain reaction-sequence-specific oligonucleotides probes, polymerase chain reaction-sequence-specific primers, sequencing-based typing, or a combination thereof.

11. The method of claim 1, wherein the step of comparing the identity between the killer-cell immunoglobulin-like receptor genes of the third test specimen and the killer-cell immunoglobulin-like receptor genes of the target gene group comprises: comparing genetic identity of 2DL1, 2DL2, 2DL3, 2DL4, 2DL5, 2DS1, 2DS2, 2DS3, 2DS4, 2DS5, 3DL1, 3DL2, 3DL3, 3DS1, 2DP1, 3DP1, or a combination thereof.

12. A method of determining identity of a plurility of source subjects of a plurility of specimens, comprising:
providing a test specimen and a target specimen;
comparing an identity between human leukocyte antigen genes of the test specimen and human leukocyte antigen genes of the target specimen to obtain a comparison result of human leukocyte antigen genes;
comparing an identity between short tandem repeats of the test specimen and short tandem repeats of the target specimen to obtain a comparison result of short tandem repeats;
comparing an identity between killer-cell immunoglobulin-like receptor genes of the test specimen and killer-cell immunoglobulin-like receptor genes of the target specimen to obtain a comparison result of killer-cell immunoglobulin-like receptor genes; and
wherein when the comparison result of human leukocyte antigen genes, the comparison result of short tandem repeats and the comparison result of killer-cell immunoglobulin-like receptor genes are all identical, a source subject of the test specimen is determined to be identical to a source subject of the target specimen.

13. The method of claim 12, wherein the test specimen and the target specimen comprise blood.

14. The method of claim 12, wherein the step of comparing the identity between the human leukocyte antigen genes of the test specimen and the human leukocyte antigen genes of the target specimen comprises:
extracting and amplifying the human leukocyte antigen genes of the test specimen and the human leukocyte antigen genes of the target specimen; and
analyzing and comparing the identity between the human leukocyte antigen genes of the test specimen and the human leukocyte antigen genes of the target specimen by using next generation sequencing.

15. The method of claim 12, wherein the step of comparing the identity between the human leukocyte antigen genes of the test specimen and the human leukocyte antigen genes of the target specimen comprises: comparing genetic identity of HLA-A, HLA-B, HLA-C, HLA-DRB1, HLA-DQB1, or a combination thereof.

16. The method of claim 12, wherein the step of comparing the identity between the human leukocyte antigen genes of the test specimen and the human leukocyte antigen genes of the target specimen comprises:
comparing an identity between a coding region of the human leukocyte antigen genes of the test specimen and a coding region of the human leukocyte antigen genes of the target specimen; and
comparing an identity between a non-coding region of the human leukocyte antigen genes of the test specimen and a non-coding region of the human leukocyte antigen genes of the target specimen.

17. The method of claim 12, wherein the step of comparing the identity between the short tandem repeats of the test specimen and the short tandem repeats of the target specimen comprises:
extracting the short tandem repeats of the test specimen and the short tandem repeats of the target specimen; and
analyzing and comparing the identity between the short tandem repeats of the test specimen and the short tandem repeats of the target specimen by using sequencing-based typing.

18. The method of claim 12, wherein the step of comparing the identity between the short tandem repeats of the test specimen and the short tandem repeats of the target specimen comprises: comparing genetic identity of gene locus D8S1179, D21S11, D7S820, CSF1PO, D3S1358, TH01, D13S317, D16S539, D2S1338, D19S433, vWA, TPOX, D18S51, Amelogenin, D5S818, FGA, or a combination thereof.

19. The method of claim 12, wherein the step of comparing the identity between the killer-cell immunoglobulin-like receptor genes of the test specimen and the killer-cell immunoglobulin-like receptor genes of the target specimen, comprising:
extracting the killer-cell immunoglobulin-like receptor genes of the test specimen and the killer-cell immunoglobulin-like receptor genes of the target specimen; and
analyzing and comparing the identity between the killer-cell immunoglobulin-like receptor genes of the test specimen and the killer-cell immunoglobulin-like receptor genes of the target specimen by using polymerase chain reaction-sequence-specific oligonucleotides probes, polymerase chain reaction-sequence-specific primers, sequencing-based typing, or a combination thereof.

20. The method of claim 12, wherein the step of comparing the identity between the killer-cell immunoglobulin-like receptor genes of the test specimen and the killer-cell immunoglobulin-like receptor genes of the target specimen comprises comparing genetic identity of 2DL1, 2DL2, 2DL3, 2DL4, 2DL5, 2DS1, 2DS2, 2DS3, 2DS4, 2DS5, 3DL1, 3DL2, 3DL3, 3DS1, 2DP1, 3DP1, or a combination thereof.
